SEQUENCE LISTING (20 sequences)

SEQ ID NO: 1 - protein, 201 aa, Unknown
  C.glutamicum pheA
MSDAPIVVAYLGPAGTFTEEALYKFADAGVFGDGEIEQLPAKSPQEAVDAVRHGTAQFAVVAIENFVDGPVTPTFDALDQGSNVQIIAEEELDIAFSIMVRPGTSLADVKTLATHPVGYQQVKNWMATTIPDAMYLSASSNGAGAQMVAEGTADAAAAPSRAAELFGLERLVDDVADVRGARTRFVAVQAQAAVSEPTGHD

SEQ ID NO: 2 - DNA, 603 bp, Unknown
  C.glutamicum pheA
atgagcgacgcaccaattgttgtggcctatttggggcctgccggaaccttcaccgaagaagccctctacaaatttgccgacgccggcgtattcggcgacggtgagatcgagcagctaccagccaaatcgccacaagaagctgtcgacgcggtccgccacggcaccgcccagttcgcggtggtcgccatcgaaaacttcgtcgacggccccgtcacccccaccttcgacgcccttgaccagggctccaacgtgcaaatcatcgccgaagaagaactcgatattgccttttccatcatggtccggccagggacttcgcttgccgacgtcaaaaccctcgccacccacccggttgggtaccaacaagtgaaaaactggatggcaaccaccattccggacgccatgtatctttcagcaagctccaacggcgccggcgcacaaatggttgccgaaggaaccgccgacgcagccgcagcgccctcccgcgcagccgaactcttcggactggaacgccttgttgatgatgtcgccgacgtccgcggcgcccgcacccgcttcgttgcagtccaagcccaagcagccgtttccgaaccgaccggccacgac

SEQ ID NO: 3 - DNA, 5803 bp, Artificial sequence
  pDCM2
  misc_feature (2895)..(2895): n is a, c, g, or t
gttcgcttgctgtccataaaaccgcccagtctagctatcgccatgtaagcccactgcaagctacctgctttctctttgcgcttgcgttttcccttgtccagatagcccagtagctgacattcatccggggtcagcaccgtttctgcggactggctttctacgtgttccgcttcctttagcagcccttgcgccctgagtgcttgcggcagcgtgaagctagcttttatcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattcgagctcggtacccggggatcctctagagtcgacctgcaggcatgcaagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttggggtgggcgaagaactccagcatgagatccccgcgctggaggatcatccagccctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctgatagaaacagaagccactggagcacctcaaaaacaccatcatacactaaatcagtaagttggcagcatcacccgacgcactttgcgccgaataaatacctgtgacggaagatcacttcgcagaataaataaatcctggtgtccctgttgataccgggaagccctgggccaacttttggcgaaaatgagacgttgatcggcacgtaagaggttccaactttcaccataatgaaataagatcactaccgggcgtattttttgagttatcgagattttcaggagctctttggcatcgtctctcgcctgtcccctcagttcagtaatttcctgcatttgcctgtttccagtcggtagatattccacaaaacagcagggaagcagcgcttttccgctgcataaccctgcttcggggtcattatagcgattttttcggtatatccatcctttttcgcacgatatacaggattttgccaaagggttcgtgtagactttccttggtgtatccaacggcgtcagccgggcaggataggtgaagtaggcccacccgcgagcgggtgttccttcttcactgtcccttattcgcacctggcggtgctcaacgggaatcctgctctgcgaggctggccggctaccgccggcgtaacagatgagggcaagcggatggctgatgaaaccaagccaaccaggaagggcagcccacctatcaaggtgtactgccttccagacgaacgaagagcgattgaggaaaaggcggcggcggccggcatgagcctgtcggcctacctgctggccgtcggccagggctacaaaatcacgggcgtcgtggactatgagcacgtccgcgagggcgtcccggaaaacgattccgaagcccaacctttcatagaaggcggcggtggaatcgaaatctcgtgatggcaggttgggcgtcgcttggtcggtcatttcgaaaaaggttaggaatacggttagccatttgcctgcttttatatagttcantatgggattcacctttatgttgataagaaataaaagaaaatgccaataggatatcggcattttcttttgcgtttttatttgttaactgttaattgtccttgttcaaggatgctgtctttgacaacagatgttttcttgcctttgatgttcagcaggaagctcggcgcaaacgttgattgtttgtctgcgtagaatcctctgtttgtcatatagcttgtaatcacgacattgtttcctttcgcttgaggtacagcgaagtgtgagtaagtaaaggttacatcgttaggcggatcaagatccatttttaacacaaggccagttttgttcagcggcttgtatgggccagttaaagaattagaaacataaccaagcatgtaaatatcgttagacgtaatgccgtcaatcgtcatttttgatccgcgggagtcagtgaacaggtaccatttgccgttcattttaaagacgttcgcgcgttcaatttcatctgttactgtgttagatgcaatcagcggtttcatcacttttttcagtgtgtaatcatcgtttagctcaatcataccgagagcgccgtttgctaactcagccgtgcgttttttatcgctttgcagaagtttttgactttcttgacggaagaatgatgtgcttttgccatagtatgctttgttaaataaagattcttcgccttggtagccatcttcagttccagtgtttgcttcaaatactaagtatttgtggcctttatcttctacgtagtgaggatctctcagcgtatggttgtcgcctgagctgtagttgccttcatcgatgaactgctgtacattttgatacgtttttccgtcaccgtcaaagattgatttataatcctctacaccgttgatgttcaaagagctgtctgatgctgatacgttaacttgtgcagttgtcagtgtttgtttgccgtaatgtttaccggagaaatcagtgtagaataaacggatttttccgtcagatgtaaatgtggctgaacctgaccattcttgtgtttggtcttttaggatagaatcatttgcatcgaatttgtcgctgtctttaaagacgcggccagcgtttttccagctgtcaatagaagtttcgccgactttttgatagaacatgtaaatcgatgtgtcatccgcatttttaggatctccggctaatgcaaagacgatgtggtagccgtgatagtttgcgacagtgccgtcagcgttttgtaatggccagctgtcccaaacgtccaggccttttgcagaagagatatttttaattgtggacgaatcaaattcagaaacttgatatttttcatttttttgctgttcagggatttgcagcatatcatggcgtgtaatatgggaaatgccgtatgtttccttatatggcttttggttcgtttctttcgcaaacgcttgagttgcgcctcctgccagcagtgcggtagtaaaggttaatactgttgcttgttttgcaaactttttgatgttcatcgttcatgtctccttttttatgtactgtgttagcggtctgcttcttccagccctcctgtttgaagatggcaagttagttacgcacaataaaaaaagacctaaaatatgtaaggggtgacgccaaagtatacactttgccctttacacattttaggtcttgcctgctttatcagtaacaaacccgcgcgatttacttttcgacctcattctattagactctcgtttggattgcaactggtctattttcctcttttgtttgatagaaaatcataaaaggatttgcagactacgggcctaaagaactaaaaaatctatctgtttcttttcattctctgtattttttatagtttctgttgcatgggcataaagttgcctttttaatcacaattcagaaaatatcataatatctcatttcactaaataatagtgaacggcaggtatatgtgatgggttaaaaaggatcaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatccgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagacagctgcgcaaggaacgcccgtcgtggccagccacgatagccgcgctgcctcgtcttggagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccg

SEQ ID NO: 4 - DNA, 366 bp, Artificial sequence
  PlysCm1
gctccttagggagccatcttttggggtgcggagcgcgatccggtgtctgaccacggtgccccatgcgattgttaatgccgatgctagggcgaaaagcacggcgagcagattgctttgcacttgattcagggtagttgactaaagagttgctcgcgaagtagcacctgtcacttttgtctcaaatattaaatcgaatatcaatatatggtctgtttattggaacgcgtcccagtggctgagacgcatccgctaaagccccaggaaccctgtgcagaaagaaaacactcctctggctaggtagacacagtttattgtggtagagttgagcgggtaactgtcagcacgtagatcgaaaggtgcacacat

SEQ ID NO: 5 - DNA, 38 bp, Artificial sequence
  Primer HR1 F
tgaattcgagctcggtacccagggtttagtgatgtccg

SEQ ID NO: 6 - DNA, 37 bp, Artificial sequence
  Primer  HR1 R
atggctccctaaggagcactgtccgcggcaagacagt

SEQ ID NO: 7 - DNA, 20 bp, Artificial sequence
  Primer HR2 F
acttgtcgactttccaggac

SEQ ID NO: 8 - DNA, 36 bp, Artificial sequence
  Primer HR2 R
gtcgactctagaggatcccccgcaacgcatgctgaa

SEQ ID NO: 9 - DNA, 37 bp, Artificial sequence
  Primer PlysCm1 F
actgtcttgccgcggacagtgctccttagggagccat

SEQ ID NO: 10 - DNA, 27 bp, Artificial sequence
  Primer PlysCm1 R
cgtcgctcatatgtgtgcacctttcga

SEQ ID NO: 11 - DNA, 27 bp, Artificial sequence
  Primer F A F
gtgcacacatatgagcgacgcaccaat

SEQ ID NO: 12 - DNA, 42 bp, Artificial sequence
  Primer F A R
gtcctggaaagtcgacaagtctagttaagtttccttccttcg

SEQ ID NO: 13 - DNA, 38 bp, Artificial sequence
  Primer UP F
tgaattcgagctcggtacccacgcacttgggtggccac

SEQ ID NO: 14 - DNA, 37 bp, Artificial sequence; identical to SEQ ID NO: 6
  Primer UP R

SEQ ID NO: 15 - DNA, 20 bp, Artificial sequence
  Primer PlysCm1 F2
acttgtcgactttccaggac

SEQ ID NO: 16 - DNA, 36 bp, Artificial sequence; identical to SEQ ID NO: 8
  Primer pheA partial R

SEQ ID NO: 17 - DNA, 16 bp, Artificial sequence
  Primer confirm_F1
ccagcgactaagcttg

SEQ ID NO: 18 - DNA, 16 bp, Artificial sequence
  Primer confirm_R1
aagccatccaagcagc

SEQ ID NO: 19 - DNA, 18 bp, Artificial sequence
  Primer confirm_F2
tctggtgcgtggttgaag

SEQ ID NO: 20 - DNA, 17 bp, Artificial sequence
  Primer  confirm_R2
tggcacattcggtaggg

(19) United States
(12) Patent Application Publication
(10) Pub. No.: US 2023/0134555 A1
(43) Pub. Date: May 4, 2023

(54) METHOD FOR PRODUCING L-TRYPTOPHAN THROUGH ENHANCEMENT OF PREPHENATE DEHYDRATASE ACTIVITY

(71) Applicant: CJ CHEILJEDANG CORPORATION, Seoul (KR)

(72) Inventors: Chang Il SEO, Seoul (KR); Hyun Ah KIM, Seoul (KR); Sung Kwang SON, Seoul (KR); Ki Yong CHEONG, Seoul (KR); Moo Young JUNG, Seoul (KR); Tae Yeon KIM, Seoul (KR)

(21) Appl. No.: 17/911,083

(22) PCT Filed: Mar. 5, 2021
(86) PCT No.: PCT/KR2021/002763

§ 371 Date: Sep. 12, 2022

(30) Foreign Application Priority Data
Mar. 17, 2020 (KR) 10-2020-0032783

(51) Int. Cl. C12N 9/88; C12P 13/22

(52) U.S. Cl. CPC C12N 9/88; C12P 13/227; C12Y 402/01051

(57) ABSTRACT

The present disclosure relates to a method for producing L-tryptophan through the enhancement of prephenate dehydratase (PheA) activity.

### TECHNICAL FIELD

The present disclosure relates to a method for producing L-tryptophan through the enhancement of prephenate dehydratase (PheA) activity.

### BACKGROUND ART

L-Tryptophan, which is one of the essential amino acids, has been widely used as a feed additive, a raw material for pharmaceuticals, such as infusions, and a material for health foods. Currently, direct fermentation using microorganisms is mainly used for the production of L-tryptophan.

As for the microorganisms used for the production of L-tryptophan, strains exhibiting resistance to L-tryptophan analogues, obtained through chemical or physical mutation, were mainly used in the early days, but recombinant strains obtained using genetic engineering techniques have been mainly used due to the rapid development of genetic recombination technology and the establishment of various molecular-level regulatory mechanisms in the 1990s.

As for the recombinant strains for producing L-tryptophan, the maximization of fermentation yield of tryptophan has been attempted by typically deleting or weakening the biosynthesis pathway of phenylalanine (Phe) or tyrosine (Tyr) on competing pathways with respect to chorismate (J Ind Microbiol Biotechnol. 2011 Dec; 38(12): 1921-9; and Appl Environ Microbiol. 1999 June; 65(6):2497-502).

However, L-tryptophan-producing strains requiring phenylalanine or tyrosine caused difficulty in having differently controlled feeding amounts of the two amino acids (phenylalanine and tyrosine) in the growth phase and production phase, an increase in additional cost in mass production, and difficulty in preparing main and feed media due to low solubility of the two amino acids (phenylalanine and tyrosine).

In order to solve such problems, the present inventors constructed a Corynebacterium strain producing L-tryptophan at high yield from wild-type Corynebacterium strains, without deleting or weakening the phenylalanine or tyrosine pathway (US 2020-0063219 A1). The use of the Corynebacterium strain producing L-tryptophan at high yield caused no accumulation of phenylalanine in a culture during high-concentration culture in a fermentation bath and produced tyrosine at a level of 0.2 g/L upon the end of culture. However, the strain produced anthranilate in the latter stage of culture and thus could not maximize the production of L-tryptophan.

### DISCLOSURE

### Technical Problem

The present inventors further optimized the metabolic flux distribution between phenylalanine and tyrosine from prephenate through the enhancement of prephenate dehydratase (PheA) activity in addition to the Corynebacterium strain producing L-tryptophan at high yield. The present inventors confirmed that the re-balance of the production of amino acids on the competing pathways controlled the final amount of phenylalanine or tyrosine produced in a culture and reduced the production of anthranilate in the latter stage of culture, and ultimately the present inventors completed the present disclosure by confirming the significant improvement in the production amount of L-tryptophan.

### Technical Solution

An aspect of the present disclosure is to provide a microorganism producing L-tryptophan and having enhanced prephenate dehydratase activity.

An aspect of the present disclosure is to provide a method for producing L-tryptophan, the method including culturing in a medium a microorganism producing L-tryptophan and having enhanced prephenate dehydratase activity.

An aspect of the present disclosure is to provide a composition for L-tryptophan production, the composition containing a microorganism producing L-tryptophan and having enhanced prephenate dehydratase activity.

### Advantageous Effects

The microorganism producing L-tryptophan and having enhanced prephenate dehydratase activity of the present disclosure can minimize the accumulation of anthranilate and produce L-tryptophan at high yield.

### BRIEF DESCRIPTION OF DRAWINGS

FIG. 1 is a schematic diagram of pDCM2 plasmid.

### BEST MODE FOR CARRYING OUT THE INVENTION

The present disclosure will be specifically described as follows. Each description and embodiment in the present disclosure may also be applied to other descriptions and embodiments. That is, all combinations of various elements in the present disclosure fall within the scope of the present disclosure. Furthermore, the scope of the present disclosure is not limited by the specific description below.

An aspect of the present disclosure is to provide a microorganism producing L-tryptophan and having enhanced prephenate dehydratase activity.

As used herein, the term “L-tryptophan” refers to one of 20 α-amino acids, which is an essential amino acid that is not biosynthesized in many organisms including humans. Tryptophan is known to mainly act as a biochemical precursor, and various substances, for example, a neurotransmitter such as serotonin, a neurohormone such as melatonin, niacin, and auxin are synthesized from tryptophan.

L-Tryptophan is synthesized from chorismate (chorismic acid), and a group of genes encoding enzymes involved in this process is known as tryptophan operon (Trp operon). The tryptophan operon is known to include structure genes and a regulatory region. A common tryptophan operon is actively transcribed so as to produce a sufficient amount of tryptophan required by cells, but when sufficient tryptophan is present in the cells, a repressor binds to tryptophan to result in inactivation of the tryptophan operon, thereby inhibiting transcription. The tryptophan operon may be derived from various microorganisms, such as a microorganism of the genus Corynebacterium and a microorganism of the genus Escherichia. The “regulatory region” of the tryptophan operon refers to a site that is present upstream of the structure genes constituting the tryptophan operon and can regulate the expression of the structure genes. The structure genes constituting the tryptophan operon in the microorganism of the genus Corynebacterium may include trpE, trpG, trpD, trpC, trpB, and trpA genes, and the structure genes constituting the tryptophan operon in the microorganism of the genus Escherichia may include trpE, trpD, trpC, trpB, and trpA genes. The regulatory region of the tryptophan operon may be present upstream of trpE at the 5′ position of the tryptophan operon structure genes. Specifically, the regulatory region of the tryptophan operon may include, a tryptophan regulator (trp regulator; trpR), a promoter (trp promoter), an operator (trp operator), a tryptophan leader peptide (trp leader peptide; trp L), and a tryptophan attenuation factor (trp attenuator), excluding the structure genes that can constitute the tryptophan operon. More specifically, the regulatory region of the tryptophan operon may include s promoter (trp promoter), an operator (trp operator), a tryptophan leader peptide (trp leader peptide; trp L), and a tryptophan attenuation factor (trp attenuator).

As used herein, the term “prephenate dehydratase” (hereinafter, “PheA”) refers to an enzyme on the pathway for production of L-phenylalanine from chorismate or prephenate, wherein the prephenate dehydratase is known as an enzyme that competes with the tyrosine biosynthesis pathway. The protein may be also named bifunctional chorismate mutase/prephenate dehydratase. An exemplary gene that encodes the protein may be pheA gene, but is not limited thereto, and the pheA gene may be regulated by the above-described tryptophan operon. Herein, the “pheA gene” may be used interchangeably with “gene encoding prephenate dehydratase” and “pheA gene”.

The PheA may have the amino acid sequence of SEQ ID NO: 1, consist of the amino acid sequence of SEQ ID NO: 1, or contain the amino acid sequence set forth in SEQ ID NO: 1, but is not limited thereto. The sequence of SEQ ID NO: 1 may be confirmed from the known database GenBank of NCBI.

Specifically, the PheA may be the amino acid sequence of SEQ ID NO: 1 and/or an amino acid sequence having at least 70%, 80%, 85%, 90%, 95%, 96%, 97%, 98%, or 99% homology or identity with SEQ ID NO: 1. It is also obvious that even PheA having an amino acid with deletion, modification, substitution, or addition in a part thereof may also be included within the scope of the present disclosure as long as the amino acid sequence has such homology or identity and exhibits activity corresponding to the PheA.

As used herein, the term “homology and identity” refers to a degree of relatedness between two given amino acid sequences or nucleotide sequences, and it may be expressed as a percentage. The terms homology and identity may be often used interchangeably.

The sequence homology or identity of conserved polynucleotides or polypeptides may be determined by standard alignment algorithms, and default gap penalties established by a program to be used may be used together. Substantially, homologous or identical sequences may be generally hybridized, under moderate or high stringent conditions, with the entire sequences or at least about 50%, 60%, 70%, 80%, or 90% of the full-lengths of the sequences. In hybridization, polynucleotides including degenerate codons instead of codons are also considered.

The homology or identity of the polypeptide or polynucleotide sequences may be determined using, for example, the algorithm BLAST by literature (see Karlin and Altschul, Pro. Natl. Acad. Sci. USA, 90, 5873(1993)) or FASTA by Pearson (see Methods Enzymol., 183, 63, 1990). Based on this algorithm BLAST, a program called BLASTN or BLASTX has been developed (see http://www.ncbi.nlm.nih.gov). In addition, the homology, similarity, or identity of any amino acid or polynucleotide sequences may be determined by comparing the sequences using southern hybridization under defined stringent conditions, and appropriate hybridization conditions to be defined may be determined within the scope of the art and by a method well known to those skilled in the art (e.g., J. Sambrook et al., Molecular Cloning, A Laboratory Manual, 2nd Edition, Cold Spring Harbor Laboratory press, Cold Spring Harbor, N.Y., 1989; and F. M. Ausubel et al., Current Protocols in Molecular Biology).

As used herein, the term “microorganism producing L-tryptophan” refers to a microorganism naturally having an L-tryptophan producing ability or a microorganism obtained by imparting L-tryptophan producing ability to a parent strain having no L-tryptophan producing ability. Specifically, the microorganism may be a microorganism producing L-tryptophan and having enhanced PheA activity, but is not limited thereto.

Specifically, the “microorganism producing L-tryptophan” includes all of wild-type microorganisms or naturally or artificially genetically modified microorganisms.

More specifically, the microorganism producing L-tryptophan may be a microorganism in which a particular mechanism is weakened or enhanced due to the insertion of an exogenous gene or the enhancement or inactivation of the activity of an endogenous gene, wherein the microorganism may be a microorganism having genetic mutation or enhanced L-tryptophan producing activity for the production of target L-tryptophan. For the purpose of the present disclosure, the microorganism producing L-tryptophan has an increase in ability to produce desired L-tryptophan through enhanced PheA activity, and such a microorganism may be a genetically modified microorganism or a recombinant microorganism, but is not limited thereto.

As used herein, the term “enhancement of activity” of a protein refers to an increase of the activity of the protein compared with intrinsic activity thereof. The “intrinsic activity” refers to the activity of a specific protein that is originally possessed by a parental strain before transformation or a non-modified microorganism when transformation occurs by genetic mutation caused by natural or artificial factors. This term may be used interchangeably with the “activity before modification”. The term “increasing” the activity of the protein compared with the intrinsic activity thereof means that the activity of the protein is enhanced compared with the activity of a particular protein originally possessed by a parent strain before transformation or a non-modified microorganism.

The “increase in activity” may be achieved by introduction of an exogenous protein or an enhancement of the activity of an endogenous protein, but may be specifically achieved by an enhancement of the activity of an endogenous protein. The enhancement or not of the activity of the protein may be confirmed by the activity degree or expression level of the corresponding protein or an increase in the amount of a product produced from the corresponding protein.

In the present disclosure, a protein to be a target of activity enhancement, i.e., a target protein, may be PheA, but is not limited thereto.

In the present disclosure, a product that is produced from the corresponding protein may be L-tryptophan, but is not limited thereto.

The enhancement of the activity of the protein may be attained by applying various methods well known in the art, and the methods are not limited as long as the methods can enhance the activity of a target protein compared with that of a microorganism before transformation. The methods may use genetic engineering or protein engineering, but are not limited thereto.

The enhancement of the activity of a protein by using genetic engineering may be implemented by, for example:

1) an increase in the intracellular copy number of the gene encoding the protein;

2) a method of replacing an expression regulatory sequence on the chromosome encoding the protein with a sequence having strong activity;

3) a method of modifying a nucleotide sequence of the initiation codon or 5′-UTR region of the protein so as to increase the activity of the protein;

4) a method of modifying a polynucleotide sequence on the chromosome so as to enhance the activity of the protein;

5) an introduction of an exogenous polynucleotide exhibiting the activity of the protein or a codon-optimized mutated polynucleotide of the polynucleotide; or 6) a combination of the methods, but are not limited thereto.

The enhancement of the activity of a protein by using protein engineering may be implemented by, for example, a method of analyzing a tertiary structure of a protein to select an exposed site and modifying or chemically modifying the site, but is not limited thereto.

The increase in the intracellular copy number of the gene encoding the protein in 1) may be performed by any method known in the art, for example, by introducing, into a host cell, a vector which is operably linked to the gene encoding the corresponding protein and can replicate and function irrespective of the host. Alternatively, such an increase may be performed by introducing, into a host cell, a vector which is operably linked to the gene and can integrate the gene into the chromosome of the host cell, but is not limited thereto.

As used herein, the term “vector” refers to a DNA construct which contains a polynucleotide sequence encoding a target protein in the form of being operably linked to an expression regulatory sequence suitable to express the target protein in a host cell. The expression regulatory sequence may include a promoter capable of initiating transcription, any operator sequence for regulating the transcription, a sequence encoding an appropriate mRNA ribosomal binding site, and sequences for regulating the termination of transcription and translation. The vector, after transformation into a suitable host cell, can replicate or function irrespective of the genome of the host, or may be integrated into the genome itself.

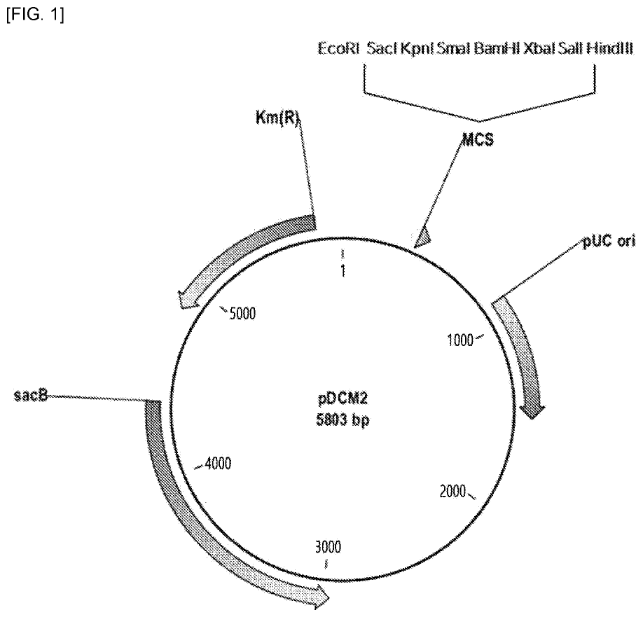

The vector used in the present disclosure is not particularly limited as long as the vector can replicate in a host cell, and any vector known in the art may be used. Examples of the vector that is commonly used may include natural or recombinant plasmids, cosmids, viruses, and bacteriophages. For example, pWE15, M13, λMBL3, λMBL4, λIXII, λASHII, λAPII, λt10, λt11, Charon4A, and Charon21A may be used as phage vectors or cosmid vectors, and pDZ-based, pBR-based, pUC-based, pBluescriptII-based, pGEM-based, pTZ-based, pCL-based, and pET-based vectors may be used as plasmid vectors. Specifically, the vector usable in the present disclosure may be pDCM2 (FIG. 1, SEQ ID NO: 3) constructed for insertion and replacement of a gene in the chromosome of Corynebacterium, but is not particularly limited thereto, and a known expression vector may be used.

As used herein, the term “transformation” indicates that a recombinant vector containing a polynucleotide encoding a target protein is introduced into a host cell to express the protein encoded by the polynucleotide in the host cell. Examples of the transformed polynucleotide may include any polypeptide that can be expressed in a host cell, regardless of whether the polypeptide is inserted and located into the chromosome of the host cell or located outside of the chromosome. A method of transformation includes any method of introducing a nucleic acid into a cell, and may be implemented by selecting a suitable standard technique known in the art depending on the host cell. Examples thereof may be electroporation, calcium phosphate (CaPO₄) precipitation, calcium chloride (CaCl₂)) precipitation, microinjection, a polyethyleneglycol (PEG) method, a DEAE—dextran method, a cationic liposome method, a lithium acetate—DMSO method, and the like, but are not limited thereto.

In addition, the term “operably linked” refers to a functional linkage between a promoter sequence or expression regulatory sequence, which initiates and mediates the transcription of the polynucleotide encoding the target protein of the present disclosure, and the polynucleotide sequence. The operable linkage may be prepared using genetic recombinant technology well known in the art, and site-specific DNA cleavage and linkage may be prepared using cleavage and linking enzymes and the like in the art, but is not limited thereto.

The method of replacing an expression regulatory sequence of the gene on the chromosome encoding the protein with a sequence having strong activity in 2) may be performed by any method known in the art, for example, by inducing sequence mutation in the nucleic acid sequence through deletion, insertion, non-conservative or conservative substitution, or a combination thereof so as to further enhance the activity of the expression regulatory sequence, or by replacement with a nucleic acid sequence having stronger activity. The expression regulatory sequence may include a promoter, an operator sequence, a sequence encoding a ribosome-binding site, sequences for regulating the termination of transcription and translation, and the like, but is not particularly limited thereto. Specifically, the method may be performed by linkage of a strong heterologous promoter instead of the original promoter, but is not limited thereto.

Known examples of the strong promoter may include mutated lysC promoter (U.S. Pat. No. 8,426,577), CJ7 promoter (U.S. Pat. No. 7,662,943 B2), CJ1 promoter (U.S. Pat. No. 7,662,943 B2), lac promoter, Trp promoter, trc promoter, tac promoter, lambda phage PR promoter, PL promoter, and tet promoter, but are not limited thereto. Specifically, a strong promoter usable in the present disclosure may be PlysCm1 (SEQ ID NO: 4) prepared by modifying a partial sequence of the mutated lysC promoter (U.S. Pat. No. 8,426,577), but is not particularly limited thereto, and a known promoter may be used.

The method of modifying a nucleotide sequence of the initiation codon or 5′-UTR region of the protein in 3) may be performed by any method known in the art, for example, by replacing the endogenous initiation codon of the protein with another initiation codon having a higher protein expression rate than the endogenous initiation codon, but is not limited thereto.

The method of modifying a polynucleotide sequence on the chromosome so as to enhance the activity of the protein in 4) may be performed by any method known in the art, for example, by inducing mutation in the nucleic acid sequence through deletion, insertion, non-conservative or conservative substitution, or a combination thereof so as to further enhance the activity of the polynucleotide sequence; or by replacement with a polynucleotide sequence which is improved to have stronger activity. The replacement may specifically be an insertion of the gene into the chromosome by homologous recombination, but is not limited thereto.

The vector used herein may further include a selection maker for checking the insertion of the chromosome. The selection marker is to select cells transformed with the vector, that is, to check whether or not a gene to be introduced is inserted, and markers providing selectable phenotypes, such as drug resistance, auxotrophy, resistance to cytotoxic agents, or surface protein expression, may be used, but are not limited thereto. Only cells that express a selection marker can survive or show a different phenotype under an environment treated with a selective agent, and thus the transformed cells can be selected.

The introduction of an exogenous polynucleotide having the activity of the protein in 5) may be performed by introducing, into a host cell, an exogenous polynucleotide encoding a protein exhibiting the same/similar activity with respect to the protein, or a codon-optimized mutant polynucleotide thereof. The exogenous polynucleotide may be used without limitation to the origin or sequence thereof as long as the polynucleotide exhibits the same/similar activity with regard to the protein. In addition, for optimized transcription and translation of the introduced exogenous polynucleotide into the host cell, a codon thereof may be optimized and introduced into the host cell. The introduction may be performed by means of any known transformation method appropriately selected by a person skilled in the art, and the introduced polynucleotide is expressed in the host cell to produce the protein, thereby enhancing the activity thereof.

Lastly, the combination of the methods in 6) may be performed by applying any one or more of 1) to 5).

Such an enhancement of the activity of the protein may indicate that the activity or concentration of the corresponding protein is increased compared with the activity or concentration of the protein expressed in a wild-type microorganism strain or a microorganism before modification, or the amount of a product produced from the corresponding protein is increased, but is not limited thereto. As used herein, the term “strain before modification” or “microorganism before modification” does not exclude a strain including mutation that may naturally occur in the microorganism, and refers to a native strain itself or a strain before trait change due to genetic mutation caused by artificial factors. In the present disclosure, the trait change may be an enhancement of the activity of PheA. The “strain before modification” or “microorganism before modification” may be used interchangeably with “non-mutated strain”, “non-modified strain”, “non-mutated microorganism”, “non-modified microorganism”, or “reference microorganism”.

In the present disclosure, the reference microorganism is not particularly limited as long as the reference microorganism produces L-tryptophan, and a mutated strain having enhanced L-tryptophan producing ability compared with a wild-type microorganism is also included without limitation. Examples thereof may include a wild-type Corynebacterium glutamicum ATCC13869 strain, a CJ04-8321 strain (WO 2019-164346 A1), or a strain in which one or more genetic modifications are added to the above strains to enhance the L-tryptophan biosynthesis pathway, but are not limited thereto.

The one or more genetic modifications may be, for example, any one or more genetic modifications selected from: overexpressing the activity of the L-tryptophan operon; improving the supply and efficiency of an L-tryptophan precursor; improving the export of L-tryptophan; attenuating or inactivating a gene on the competitive pathway, a regulator on the directional pathway of the tryptophan operon, a gene for introducing L-tryptophan, and genes for introducing and degrading tryptophan, but are not limited thereto.

The genetic modification of overexpressing the activity of the L-tryptophan operon may be, for example, i) enhancing a promoter of the L-tryptophan biosynthesis gene operon, ii) solving the feedback inhibition of the TrpE protein according to an improvement in production in the L-tryptophan operon, and iii) enhancing a promoter of the L-tryptophan biosynthesis gene operon, and specifically, the i) may be an enhancement by replacement of a promoter in the L-tryptophan biosynthesis gene operon with SPL7, a strong promoter; the ii) may be an introduction of trpE(P21S)DCBA or trpE(S38R)DCBA, which is an L-tryptophan operon having feedback inhibition trpE traits; and the iii) may be an enhancement by replacement of a promoter in the L-tryptophan biosynthesis gene operon with SPL7, a strong promoter, but is not limited thereto.

The genetic modification of improving the supply and efficiency of an L-tryptophan precursor may be, for example, an enhancement of the expression of a related gene for the continuous supply of the L-tryptophan precursor, such as erythorse-4-phosphate (E4P), and the efficient use of energy, and specifically an introduction of a gene encoding transketolase (tkt) or an enhancement of the expression thereof, but is not limited thereto.

The genetic modification of improving the export of L-tryptophan may be, for example, an introduction of an exogenous membrane protein that improves the export of L-tryptophan, and specifically an introduction of a gene (Accession number NZ_LFLU01000012.1) encoding a membrane protein derived from Herbaspirillum rhizosphaerae, but is not limited thereto.

The strain having at least one genetic modification may be, for example, CA04-8325 constructed by introducing trpE(S38R)DCBA, an L-tryptophan operon containing SPL7 as a strong promoter and having a feedback restriction trpE trait, into the ATCC13869 strain (US 2020-0063219 A1); CA04-8352 in which the tkt gene is inserted into the CA04-8325 strain (WO 2019-164346 A1), or CA04-8405 strain constructed by introducing a gene encoding a membrane protein derived from Herbaspilium risospere into the CJ04-8352 strain (US 2020-0063219 A1), but is not limited thereto.

For the purpose of the present disclosure, any microorganism producing L-tryptophan may be possible as long as the microorganism can produce L-tryptophan by an enhancement of PheA activity. Herein, the “microorganism producing L-tryptophan” may be used interchangeably with “L-tryptophan-producing microorganism” or “microorganism having L-tryptophan producing ability”.

Examples of the microorganism may include microorganisms belonging to the genera Corynebacterium, Escherichia, Enterobacter, Erwinia, Serratia, Providencia, and Brevibacterium, and specifically may be a microorganism of the genus Corynebacterium.

More specifically, the microorganism of the genus Corynebacterium may be Corynebacterium glutamicum, Corynebacterium ammoniagenes, Corynebacterium crudilactis, Corynebacterium deserti, Corynebacterium efficiens, Corynebacterium callunae, Corynebacterium stationis, Corynebacterium singulare, Corynebacterium halotolerans, Corynebacterium striatum, Corynebacterium pollutisoli, Cxorynebacterium imitans, Corynebacterium testudinoris, Corynebacterium flavescens, or the like, and the microorganism of the genus Corynebacterium may be Corynebacterium glutamicum. In addition, any microorganism belonging to the genus Corynebacterium may be included without limitation.

In accordance with another aspect of the present disclosure, there is provided a method for producing L-tryptophan, the method including culturing, in a medium, a microorganism producing L-tryptophan and having enhanced prephenate dehydratase activity.

The prephenate dehydratase, enhancement of activity, and microorganism producing L-tryptophan are as described above.

In the method, the culturing of the microorganism may be performed by known bath culturing, continuous culturing, fed-batch culturing, or the like, but is not particularly limited thereto. The culturing conditions are not particularly limited, but an appropriate pH (e.g., pH 5-9, specifically pH 6-8, and most specifically pH 7.0) may be adjusted using a basic compound (e.g., sodium hydroxide, potassium hydroxide, or ammonia) or an acidic compound (e.g., phosphoric acid or sulfuric acid), and an aerobic condition may be maintained by introduction of oxygen or an oxygen-containing gas mixture into a culture. The culturing temperature may be maintained at 20-45° C., and specifically 25-40° C., and the culturing may be performed for about 10-160 hours, but are not limited thereto. The amino acid produced by the culturing may be released into the medium or may remain in cells.

In the medium for culturing, sugars and carbohydrates (e.g., glucose, sucrose, lactose, fructose, maltose, molasses, starch, and cellulose), oils and fats (e.g., soybean oil, sunflower seed oil, peanut oil, and coconut oil), fatty acids (e.g., palmitic acid, stearic acid, and linoleic acid), alcohols (e.g., glycerol and ethanol), organic acids (e.g., acetic acid), and the like may be used separately or in mixture as carbon sources, but the carbon sources are not limited thereto. As nitrogen sources, nitrogen-containing organic compounds (e.g., peptone, yeast extract, meat extract, malt extract, corn steep liquor, soybean flour, and urea) or inorganic compound (e.g., ammonium sulfate, ammonium chloride, ammonium phosphate, ammonium carbonate, and ammonium nitrate), and the like may be used alone or in a mixture, but the nitrogen sources are not limited thereto. As phosphorus sources, potassium dihydrogen phosphate, dipotassium hydrogen phosphate, a sodium-containing salt corresponding thereto, and the like may be used alone or in a mixture, but the phosphorus sources are not limited thereto. The medium may also contain essential growth-promoting substances, such as other metal salts (e.g., magnesium sulfate or iron sulfate), amino acids, and vitamins.

The method according to the present disclosure may further include recovering L-tryptophan from the cultured medium or microorganism. The recovering of the amino acid produced in the culturing step may be collecting a target amino acid from the culture by using an appropriate method known in the art according to the culturing method. For example, centrifugation, filtration, anion exchange chromatography, crystallization, HPLC, and the like may be used, and the target amino acid can be recovered from the medium or microorganism by using an appropriate method known in the art.

The recovering step may include a purification process, which may be performed using an appropriate method known in the art. Therefore, the recovered amino acid may be in a purified form or may be a microorganism fermentation liquid containing an amino acid (Introduction to Biotechnology and Genetic Engineering, A. J. Nair, 2008). The recovering of the target amino acid can be efficiently performed by adding a suitable method known in the art before and after the culturing step or before and after the recovering step.

In accordance with another aspect of the present disclosure, there is provided a composition for L-tryptophan production, the composition containing a microorganism producing L-tryptophan and having enhanced prephenate dehydratase activity.

The prephenate dehydratase, enhancement of activity, and microorganism producing L-tryptophan are as described above.

The composition for L-tryptophan production may contain pheA gene encoding the PheA, and may include, without limitation, an element that can enhance the PheA or pheA gene. Specifically, the element may be in the form of being contained in a vector so as to express a gene operably linked to an introduced host cell, and the form is as described above. Specifically, the expression of pheA gene, which is a gene encoding prephenate dehydratase, may be enhanced through an increase in gene copy number or a replacement with a strong promoter.

In accordance with still another aspect of the present disclosure, there is provided use of a composition for producing L-tryptophan.

### MODE FOR CARRYING OUT THE INVENTION

Hereinafter, the present disclosure will be described in more detail with reference to exemplary embodiments. However, these exemplary embodiments are given for specifically illustrating the present disclosure, and the scope of the present disclosure is not limited to these exemplary embodiments.

### Example 1: Construction of Plasmid

A plasmid (pDCM2, FIG. 1, SEQ ID NO: 3) for the insertion and replacement of a gene in the Corynebacterium chromosome was designed. The plasmid was synthesized using the Gene-synthesis service by Bionix Co., Ltd. The plasmid was designed to include a restriction enzyme that can be easily utilized in cloning with reference to a paper associated with the generally known sacB system (Gene, 145 (1994) 69-73). The pDCM2 plasmid thus synthesized has the following characteristics.

1) The plasmid has a replication origin that functions only in E. coli, and thus the plasmid can attain self-replication thereof in E. coli but cannot attain self-replication thereof in Corynebacterium.

2) The plasmid has a kanamycin resistance gene as a selection marker. 3) The plasmid has the levansucrose gene (sacB) as a secondary positive-selection marker.

4) No genetic information derived from the pDCM2 plasmid is left in the finally constructed strain.

### Example 2: Construction of Plasmid for Enhancement of Prephenate Dehydratase

To enhance the activity of prephenate dehydratase (hereinafter, “pheA”), SEQ ID NO: 4 was designed by modifying a partial sequence on the basis of a mutated lysC promoter (U.S. Pat. No. 8,426,577 B2) known to be a strong promoter, synthesized using the Gene-synthesis service by Bionix Co., Ltd., and named PlysCm1. The PlysCm1 promoter was used to construct a plasmid for enhancing prephenate dehydratase activity through the additional insertion of pheA gene or replacement of the wild-type promoter of the pheA gene with PlysCm1.

### Example 2-1: Construction of Plasmid for Gene Insertion

To further insert pheA gene with the PlysCm1 promoter, the upstream and downstream regions for homologous recombination on the chromosome were amplified using wild-type Corynebacterium glutamicum ATCC13869 chromosomal DNA as a template along with the primer pair of SEQ ID NO: 5 and SEQ ID NO: 6 and the primer pair of SEQ ID NO: 7 and SEQ ID NO: 8, thereby obtaining gene fragments, respectively. The primer sequences used herein are shown in Table 1 below.

TABLE 1

| SEQ | | |
|---|---|---|
| ID | | |
| NO | Name | Sequence (5′→3′) |
| 5 | HR1 F | tgaattcgagctcggtacccAGGGTTTAGTGATGTCCG |
| 6 | HR1 R | ATGGCTCCCTAAGGAGCACTGTCCGCGGCAAGACAGT |
| 7 | HR2 F | ACTTGTCGACTTTCCAGGAC |
| 8 | HR2 R | gtcgactctagaggatccccCGCAACGCATGCTGAA |

PCR was performed to obtain the fragments. Solg™ Pfu-X DNA polymerase was used as a polymerase, and PCR amplification was performed under conditions: denaturation at 95° C. for 4 minutes, 30 cycles of denaturation at 95° C. for 30 seconds, annealing at 60° C. for 30 seconds, and polymerization at 72° C. for 50 seconds, and then extension at 72° C. for 5 minutes.

A PlysCm1 promoter fragment was obtained using the previously synthesized SEQ ID NO: 4 as a template along with SEQ ID NO: 9 and SEQ ID NO: 10. Additionally, a pheA gene fragment (SEQ ID NO: 2) was obtained using the wild-type Corynebacterium glutamicum ATCC13869 chromosomal DNA as a template along with SEQ ID NO: 11 and SEQ ID NO: 12. The primer sequences used herein are shown in Table 2 below.

TABLE 2

| SEQ | | |
|---|---|---|
| ID | | |
| NO | Name | Sequence (5′→3′) |
| 9 | PlysCm1 F | ACTGTCTTGCCGCGGACAGTGCTCCTTAGGGAG |
| | | CCAT |
| 10 | PlysCm1 R | CGTCGCTCATATGTGTGCACCTTTCGA |
| 11 | PheA F | GTGCACACATATGAGCGACGCACCAAT |
| 12 | PheA R | GTCCTGGAAAGTCGACAAGTCTAGTTAAGTTTC |
| | | CTTCCTTCG |

Solg™ Pfu-X DNA polymerase was used as a polymerase, and PCR amplification was performed under conditions: denaturation at 95° C. for 4 minutes, 27 cycles of denaturation at 95° C. for 30 seconds, annealing at 60° C. for 30 seconds, and polymerization at 72° C. for 1 minute, and then extension at 72° C. for 5 minutes.

The upstream and downstream fragments for homologous recombination on the chromosome, the PlysCm1 promoter fragment, the pheA gene fragment, and the pDCM2 vector for chromosomal transformation cleaved by Smal restriction enzyme, which were obtained the above processes, were cloned by the Gibson assembly method (D. G. Gibson et al., NATURE METHODS, Vol. 6 No. 5, May 2009, NEBuilder HiFi DNA Assembly Master Mix) to obtain a recombinant plasmid, which was then named pDCM2-Tn::PlysCm1_pheA.

### Example 2-2: Construction of Plasmid for Promoter Replacement

The construction of a plasmid having enhanced prephenate dehydratase activity was attempted by replacing the wild-type promoter of pheA gene with PlysCm1. Specifically, a gene fragment of the upstream region of the wild-type promoter for pheA gene for homologous recombination on the chromosome was obtained using wild-type Corynebacterium glutamicum (ATCC13869) chromosomal DNA as a template along with the primer pair of SEQ ID NO: 13 and SEQ ID NO: 14. In addition, a gene fragment containing both the PlysCm1 promoter and the downstream thereof was obtained using the previously constructed pDCM2-Tn::PlysCm1_pheA plasmid as a template along with SEQ ID NO: 15 and SEQ ID NO: 16. The primer sequences used herein are shown in Table 3 below.

TABLE 3

| SEQ | | |
|---|---|---|
| ID | | |
| NO | Name | Sequence (5′→3′) |
| 13 | UP F | tgaattcgagctcggtacccACGCACTTGGGTG |
| | | GCCAC |
| 14 | UP R | ATGGCTCCCTAAGGAGCACTGTCCGCGGCAAG |
| | | ACAGT |
| 15 | PlysCm1 | ACTTGTCGACTTTCCAGGAC |
| | F2 | |
| 16 | pheA | gtcgactctagaggatccccCGCAACGCATGCT |
| | partialR | GAA |

To obtain the above fragments, Solg™ Pfu-X DNA polymerase was used as a polymerase, and PCR amplification was performed under conditions: denaturation at 95° C. for 4 minutes, 27 cycles of denaturation at 95° C. for 30 seconds, annealing at 60° C. for 30 seconds, and polymerization at 72° C. for 50 seconds, and then extension at 72° C. for 5 minutes.

The upstream fragment of the pheA promoter, the promoter and downstream fragment containing PlysCm1, and the pDCM2 vector for chromosomal transformation cleaved by Smal restriction enzyme, which were obtained the above processes, were cloned by the Gibson assembly method to obtain a recombinant plasmid, which was then named pDCM2-Pn::PlysCm1_pheA.

### Example 3: Construction of Strain Having Enhanced Prephenate Dehydratase Activity and Production of Tryptophan

pDCM2-Tn::PlysCm1_pheA constructed in Example 2-1 was transformed, through electroporation (Appl. Microbiol. Biotechnol. (1999) 52:541-545), into the CA04-8405 strain (KCCM12099P, US 2020-0063219 A1) constructed by introduction of a gene (Accession number NZ_LFLU01000012.1) encoding a membrane protein derived from Herbaspirillum rhizosphaerae into the CA04-8352 strain (Korean Patent No. 10-1968317), followed by secondary crossover, thereby obtaining a strain in which the PlysCm1_pheA gene was further inserted. The insertion of the corresponding gene was confirmed through PCR amplification and genome sequencing using the primer pair of SEQ ID NO: 17 and SEQ ID NO: 18 capable of amplifying the external sites of the upstream and downstream regions of the corresponding homologous recombination. The strain with the gene inserted was named CM05-9157. The primer sequences used herein are shown in Table 4 below.

TABLE 4

| SEQ ID NO | Name | Sequence (5′→3′) |
|---|---|---|
| 17 | confirm_F1 | CCAGCGACTAAGCTTG |
| 18 | confirm_R1 | AAGCCATCCAAGCAGC |

The pDCM2-Pn::PlysCm1_pheA constructed in Example 2-2 was transformed into CA04-8405 strain by using electroporation according to the same method as above, followed by secondary crossover, thereby obtaining a strain in which the wild-type pheA promoter was replaced with the PlysCm1 promoter. The replacement of the promoter was confirmed through PCR amplification and genome sequencing using the primer pair of SEQ ID NO: 19 and SEQ ID NO: 20 capable of amplifying the external sites of the upstream and downstream regions of the corresponding homologous recombination. The strain with the promoter replaced was named CM05-9158. The primer sequences used herein are shown in Table 5 below.

TABLE 5

| SEQ ID NO | Name | Sequence (5′→3′) |
|---|---|---|
| 19 | confirm_F2 | TCTGGTGCGTGGTTGAAG |
| 20 | confirm_R2 | TGGCACATTCGGTAGGG |

To investigate the production of tryptophan by the CM05-9157 and CM05-9158 strains constructed through the above processes, culturing was conducted by the same method as below and the production amount of tryptophan were compared with that by the CA04-8405 strain as a control. Each strain was inoculated into a 250 mL corner-baffle flask containing 25 mL of a seed medium, and cultured with shaking at 200 rpm for 20 hours at 30° C. After the culturing, 1 mL of the seed culture was inoculated into a 250 mL corner-baffle flask containing 25 mL of a production medium prepared in triplicate for each strain, and cultured with shaking at 200 rpm for 24 hours at 30° C. After the completion of culturing with shaking, the production amount of L-tryptophan was measured using HPLC.

Seed Medium (pH 7.0)

glucose 20 g, peptone 10 g, yeast extract 5 g, urea 1.5 g, KH₂PO₄ 4 g, K₂HPO₄ 8 g, MgSO₄ 7H₂O 0.5 g, biotin 100 μg, thiamine HCl 1000 μg, calcium pantothenate 2000 μg, nicotinamide 2000 μg (based on 1 L of distilled water)

Production Medium (pH 7.0)

Glucose 30 g, (NH₄)₂SO₄ 15 g, MgSO₄ 7H₂O 1.2 g, KH₂PO₄ 1 g, yeast extract 5 g, biotin 900 μg, thiamine hydrochloride 4500 μg, calcium pantothenate 4500 μg, CaCO₃ 30 g (based on 1 L of distilled water).

The results of the L-tryptophan production by the CA04-8405 strain and the CM05-9157 and CM05-9158 strains with enhanced pheA expression in the medium are shown in Table 6 below.

TABLE 6

| | | L- | Tryptophan | |
|---|---|---|---|---|
| | | Tryptophan | yield | Anthranilate |
| | OD₅₆₂ | (g/L) | (*100 g/g, %) | (g/L) |
| | (Standard | (Standard | (Standard | (Standard |
| | deviation) | deviation) | deviation) | deviation) |
| CA04-8405 | 53.2 (0.82) | 1.57 (0.03) | 5.22 (0.11) | 0.17 (0.01) |
| CM05-9157 | 56.5 (0.45) | 1.93 (0.02) | 6.43 (0.07) | 0.00 |
| CM05-9158 | 56.4 (0.08) | 1.94 (0.02) | 6.48 (0.06) | 0.00 |

As a result of culturing of the CM05-9157 and CM05-9158 strains with enhanced pheA expression, the strains produced L-tryptophan at 1.93 g/L and 1.94 g/L, respectively. These results indicate an increase of about 0.37 g/L and an improvement in fermentation yield of about 23-24% compared with the control CA04-8405 strain. It was also confirmed that the production of anthranilate was reduced due to the enhancement of pheA expression, which led to an increase in the production amount of tryptophan.

The CM05-9157 strain was internationally deposited at the Korean Culture Center of Microorganisms (KCCM), an international depositary, on 20 Feb. 2020, under the provisions of the Budapest Treaty, and assigned Accession number KCCM12670P.

As set forth above, a person skilled in the art to which the present disclosure pertains will be able to understand that the present disclosure may be embodied in other specific forms without departing from the technical spirit or essential characteristics thereof. Therefore, the embodiments described above should be construed as being exemplified and not limiting the present disclosure. The scope of the present disclosure should be understood that all changes or modifications derived from the definitions and scopes of the claims and their equivalents fall within the scope of the disclosure.

1. A microorganism producing L-tryptophan and having enhanced prephenate dehydratase activity.

2. The microorganism of claim 1, wherein the prephenate dehydratase contains the amino acid sequence of SEQ ID NO: 1 or an amino acid sequence having at least 90% sequence identity therewith.

3. The microorganism of claim 1, wherein the enhanced activity is obtained by an increase in copy number of a gene encoding the prephenate dehydratase or a replacement of a promoter of the gene with a strong promoter.

4. The microorganism of claim 1, wherein the microorganism is Corynebacterium sp.

5. The microorganism of claim 4, wherein the microorganism is Corynebacterium glutamicum.

6. A method for producing L-tryptophan, the method comprising culturing a microorganism producing L-tryptophan and having enhanced prephenate dehydratase activity.

7. The method of claim 6, further comprising recovering L-tryptophan from the cultured medium or microorganism.

8. A composition for L-tryptophan production, the composition comprising a microorganism producing L-tryptophan and having enhanced prephenate dehydratase activity.
